(19) United States
(12) Patent Application Publication
(10) Pub. No.: US 2022/0355032 A1
(43) Pub. Date: Nov. 10, 2022

(54) A SYSTEM WITH A MONITORING DEVICE

(71) Applicant: QUIO TECHNOLOGIES LLC, NEW YORK, NY (US)

(72) Inventor: Jared SCHWARTZENTRUBER, New York, NY (US)

(21) Appl. No.: 17/621,329

(22) PCT Filed: Jun. 23, 2020
(86) PCT No.: PCT/IB2020/055903

§ 371 Date: Dec. 21, 2021

Related U.S. Application Data
(60) Provisional application No. 62/866,563, filed on Jun. 25, 2019.

(30) Foreign Application Priority Data
Oct. 7, 2019 (EP) 19201661.6

(51) Int. Cl. A61M 5/20; A61M 5/315

(52) U.S. Cl. CPC A61M 5/20; A61M 5/31501

(57) ABSTRACT

A system having a monitoring device and a medicament delivery device is presented wherein the monitoring device is configured to be releasably attached to the medicament delivery device and to monitor an operation of the medicament delivery device. The monitoring device has a body defining a cavity for receiving the medicament delivery device; a biased holding unit arranged in the body and configured to retain the medicament delivery device to the body in a secure state; wherein the biased b holding unit is movable within the body between a first position in which the medicament delivery device is in an unsecure state and a second position in which the medicament delivery device is in the secure state. The biased holding unit can have an enclosing member having a engagement structure; and in that the biased holding unit there can be a locking element fixed to or integral with the enclosing member, wherein the engagement structure is configured to interact with an engaging member on the medicament delivery device and the locking element is configured to interact with a counter locking element arranged to the body when the biased holding unit is in the second position.

### CROSS REFERENCE TO RELATED APPLICATIONS

The present application is a U.S. National Phase Application pursuant to 35 U.S.C. § 371 of International Application No. PCT/M2020/055903 filed Jun. 23, 2020, which claims priority to U.S. Provisional Patent Application No. 62/866,563 filed Jun. 25, 2019, and European Patent Application No. 19201661.6 filed Oct. 7, 2019. The entire disclosure contents of these applications are herewith incorporated by reference into the present application.

### TECHNICAL FIELD

The present disclosure generally relates to a system with a monitoring device. In particular, it relates to a monitoring device for releasably attaching to a self-medicament delivery device.

### BACKGROUND

Medicament delivery devices such as auto-injectors, inhalers, on-body devices are generally known for the self-administration of a medicament by patients without formal medical training. As just one example, patients suffering from diabetes may require repeated injections of insulin. Other patients may require regular injections of other types of medicaments, such as a growth hormone. Reusable auto-injectors are commonly used for patients who are required to apply repeated injections.

There is an ever-increasing demand for monitoring the handling of medicament delivery devices. One important reason for monitoring the handling is to provide the user with information and alerts as to when the next dose delivery is scheduled, based on previous dose deliveries and based on prescribed medication schedules.

Some medicament delivery devices are provided with monitoring units integrated into the medicament delivery device. This has a number of drawbacks. One is that the medicament delivery device becomes rather complicated and expensive to manufacture. This is especially a problem if the medicament delivery device is a so-called disposable device that is to be discarded after use. Another drawback is that it is not a simple task to modify a medicament delivery device that already is on the market by integrating monitoring functionality, because such modifications require new approvals from national health and/or regulatory authorities before they can be marketed.

An alternative is to provide monitoring units that can be releasably connected to a medicament delivery device. This solution has a number of advantages. First, the monitoring unit may be used many times which is an advantage when disposable medicament delivery devices are used. It is then easy to remove the monitoring unit from a used medicament delivery device that is to be discarded and to connect it to a new medicament delivery device for the subsequent dose delivery. Moreover, the monitoring units may be easily adapted or modified to be connected to medicament delivery devices on the market without the medicament delivery devices being affected regarding design and function by the monitoring units, thereby omitting any intervention by the national health and/or regulatory authorities.

WO2013/120774 discloses a supplemental device for attachment to an injection device through clipping on or hinge clipping arm connection, which the injection device and the supplemental device moving closer together, the force results in the arms being moved apart, against the resilience of the spring. This causes the spring to apply a reaction force, which resists entry of the injection device into the injection device receiving channel, once the injection device reach a certain position, the reaction force supplied by the spring ceases to increase upon further movement of the injection device and the supplemental device together, then the movement of the injection pen into the injection device receiving channel is aided by the resilience of the spring; or the latching edge on an resilient closure engage a latching face to lock the injection device in a passage defined by the hook, and when the force applied is sufficient, the closure will deform to a point where the latching edge is no longer engaged with the latch engaging face.

However, the attaching mechanism disclosed in WO2013/120774 requires a certain force to overcome an abutment for either to lock the injection device to the supplemental device or release from the supplemental device, which abutment is for securing the injection device with the supplemental device, so for tightly securing the injection device with the supplemental device, the certain force for overcoming the abutment will be high, which is difficult for some users to handle, such like the elderly, children or the patient with rheumatoid arthritis.

### SUMMARY

An object of the present disclosure is to provide an activation mechanism for an automatic medicament delivery device which solves problems of the prior art.

In the present disclosure, when the term “distal” is used, this refers to the direction pointing away from the dose delivery site. When the term “distal part/end” is used, this refers to the part/end of the delivery device, or the parts/ends of the members thereof, which under use of the medicament delivery device is/are located furthest away from the dose delivery site. Correspondingly, when the term “proximal” is used, this refers to the direction pointing to the dose delivery site. When the term “proximal part/end” is used, this refers to the part/end of the delivery device, or the parts/ends of the members thereof, which under use of the medicament delivery device is/are located closest to the dose delivery site.

Further, the term “longitudinal” or “axial”, refers to a direction extending from the proximal end to the distal end and through the device or components thereof in the direction if the longest extension of the device and/or component.

Similarly, the terms “traverse” refers to a direction generally perpendicular to the longitudinal direction.

An object of this disclosure is to provide a simple and reliable way of attaching and securing a monitoring device to a medicament delivery device, especially a self-administration device; the monitoring is configured to detect a medicament delivery process, such as, at least one of the priming operation, the delivery start point, the delivery operation and/or the end of delivery of a self-administration device. The object of this disclosure may be employed for monitoring the use or handling of fixed dose delivery devices including single-dose delivery devices such as auto-injectors or multi-dose injection device, inhalers, on-body devices, infusion devices, or medical sprayers.

According to an aspect of this disclosure, the object is achieved by a medicament delivery device according to claim 1.

There is hence provided a system comprising a monitoring device and a medicament delivery device wherein the monitoring device is configured to be releasably attached to the medicament delivery device and to monitor an operation of the medicament delivery device, wherein the monitoring device comprises: a body defining a cavity for receiving the medicament delivery device; a biased holding unit arranged in the body and configured to retain the medicament delivery device to the body in a secure state; the biased holding unit is movable within the body between a first position in which the medicament delivery device is in an unsecure state and a second position in which the medicament delivery device is in the secure state; the biased holding unit comprises an enclosing member having an engagement structure; and in that the holding unit further comprises a locking element fixed to or integral with the enclosing member, the engagement structure is configured to interact with an engagement member on the medicament delivery device and the locking element is configured to interact with a counter locking element arranged to the body when the biased holding unit is in the second position.

According to one embodiment a biasing element is positioned between the body and the medicament delivery device.

According to one embodiment the biasing element can engage or integral with the biased holding unit at one end and engaged or integral the body of the monitoring device at the other end; or engage the medicament delivery device at one end, and engaged or integral the body of the monitoring device at the other end.

According to one embodiment the biasing element is configured to be in a compressed state when the biased holding unit is in the second position, and in a released state when the biased holding unit is in the first position.

According to one embodiment the biasing element can be a coil metal spring, a plastic spring or a flexible arm.

According to one embodiment the enclosing member can be a ring shape, a rectangular shape or a triangle shape element; depending on the shape of the housing of the medicament delivery device.

According to one embodiment the engagement structure is a flexible arm configured to be fixedly engaged with engaging member on the medicament delivery device when the medicament delivery device is in the secure state.

According to one embodiment the engagement structure can also be alternatively as see-saw element.

According to one embodiment the engagement structure can also be alternatively as a rotationally engagement element with a first rotational position, which the rotationally engagement element is engaged with the medicament delivery device; and a second rotational position, which the rotationally engagement element is disengaged from the medicament delivery device.

According to one embodiment the engagement member can be an extra design on the medicament delivery device, such like a fixture rib; or an existing part of the medicament delivery device, such like an edge of the observed window of the medicament delivery device; or the surface of the housing of the medicament delivery device.

According to one embodiment the body comprises a movable portion.

According to one embodiment the movable portion can be moved by a user with a twisting movement, an axial movement or a traverse movement in relation to a longitudinal axis.

According to one embodiment the counter locking element is arranged to the movable portion of the body.

According to one embodiment the counter locking element is arranged on the inner surface of the movable portion of the body.

According to one embodiment the biased holding unit is rotationally locked and slidable in relation to the body between the first position and the second position.

According to one embodiment the engagement structure is an axial stop rib configured to interact with the engaging member on the medicament delivery device for axially locking the medicament delivery device with the biased holding unit.

According to one embodiment the engagement structure is a radial stop rib configured to interact with the engaging member on the medicament delivery device for radially locking the medicament delivery device with the biased holding unit.

According to one embodiment the axial stop rib is a wedge shape structure.

According to one embodiment the medicament delivery device can be an injection device, an infusion device, an on-body device an inhalation device or a medical sprayer.

According to one embodiment, the monitoring device comprises an electronic model configured to detect at least one of a priming operation, a dose dial operation, a medicament delivery operation or an end of medicament delivery of the medicament delivery device.

According to one embodiment, the body of the monitoring device is configured to either enclose the medicament delivery device on the distal end of the medicament delivery device or receiving the medicament delivery device with the distal end and the proximal end of the medicament delivery device are both protruding from the body of the monitoring device.

Other aspects, features, and advantages will be apparent from the summary above, as well as from the description that follows, including the figures and the claims.

Generally, all terms used in the claims are to be interpreted according to their ordinary meaning in the technical field, unless explicitly defined otherwise herein. All references to “a/an/the element, apparatus, component, means, etc. are to be interpreted openly as referring to at least one instance of the element, apparatus, component, means, etc.”, unless explicitly stated otherwise.

### BRIEF DESCRIPTION OF THE DRAWINGS

The specific embodiments of the objects of this disclosure concept will now be described, by way of example, with reference to the accompanying drawings, in which:

FIG. 1 displays an exploded view of the monitoring device.

FIG. 2 displays the system wherein the monitoring device is attached to a medicament delivery device.

FIGS. 3 and 4 display perspective cross sectional view of the biased holding unit.

FIGS. 5 and 6 display a perspective view of the system when the medicament delivery device is being positioned in the cavity of the monitoring unit and when the medicament delivery device is positioned and secured in the cavity of the monitoring unit.

FIG. 7 displays a perspective view of the system when the medicament delivery device is to be released from the monitoring device.

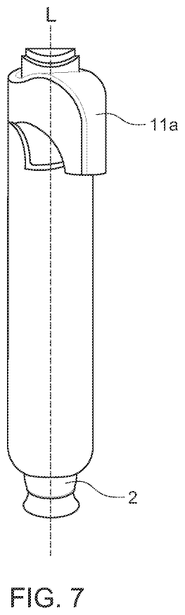

### DETAILED DESCRIPTION

The concept of this disclosure will now be described more fully hereinafter with reference to the accompanying drawings, in which exemplifying embodiments are shown. The concept of this disclosure may, however, be embodied in many different forms and should not be construed as limited to the embodiments set forth herein; rather, these embodiments are provided by way of example so that this disclosure will be thorough and complete, and will fully convey the scope of the concept of this disclosure to those skilled in the art. Like numbers refer to like elements throughout the description.

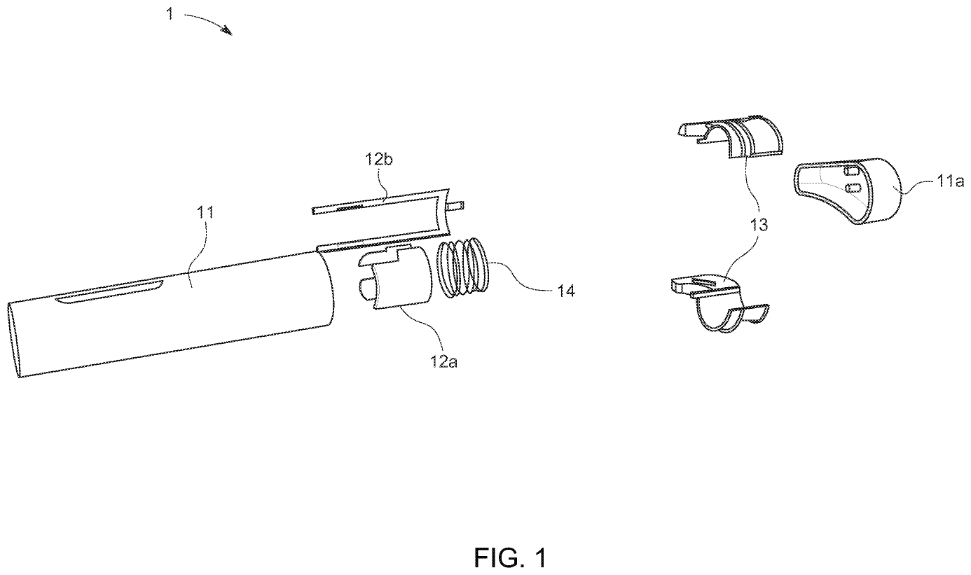

FIG. 1 illustrate an exploded view of the monitoring device. The monitoring device (1) comprises a body (11) defining a cavity which extends along a longitudinal axis L and which is configured to receive a medicament delivery device (2); and a biased holding unit (12) positioned inside the cavity. The biased holding unit (12) comprises an enclosing member (12a) and, a locking element (12b). In a preferred embodiment, the system further comprises a biasing element (14) arranged between the biased holding unit (12) and the body. In a preferred embodiment, the biasing element is a compression spring arranged to abut the enclosing member (12a) at one end, and an inner protruding ledge on the inner surface of the body on the other end. The body (11) further comprises a movable portion (11a) which is positioned at its distal end and a counter locking element (13). In the preferred embodiment the movable portion is twistable in relation to the longitudinal axis (L). It is also possible that in a second embodiment the movable portion is moved along the longitudinal axis L when a user pulls the movable portion. It is also obvious that a translational movement can be implemented, i.e. by rotating and pulling.

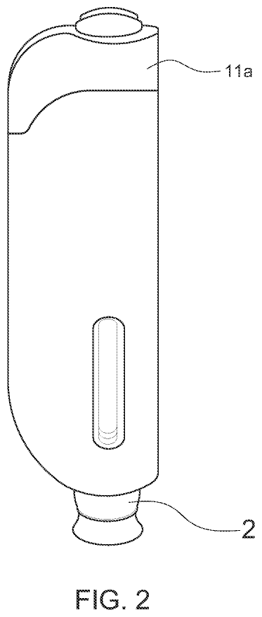

FIG. 2 illustrates the system where the monitoring device receives the medicament delivery device (2) such that the distal and the proximal end of the medicament delivery device (2) protrudes from the respective distal and proximal ends of the body (11). In another embodiment, the body can be configured to enclose the distal end of the medicament delivery device (2) and expose the proximal end of the medicament delivery device (2). Embodiments where the medicament delivery device (2) comprises a trigger button or a dose dial knob on its distal end, then the preferable body shall be the first mentioned.

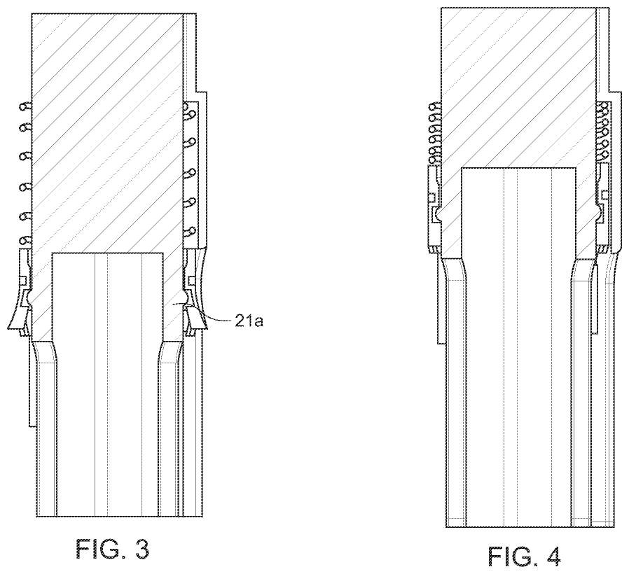

FIG. 3 illustrates a cross sectional view of the biased holding unit in a first position in which the medicament delivery device is in an unsecure state and the biasing element (14) is in a released state. FIG. 3 also illustrates that the medicament delivery device comprises an engagement member (21a) that can be an extra design on the outer surface of the medicament delivery device, such as a fixture rib, a protrusion, a hole; or an existing part of the medicament delivery device, such like an edge of the observation window on the outer surface of the medicament delivery device.

FIG. 4 illustrates a cross sectional view of the biased holding unit (12) in a second position in which the medicament delivery device (2) is in a secure state and the biasing element (14) is in a compressed state.

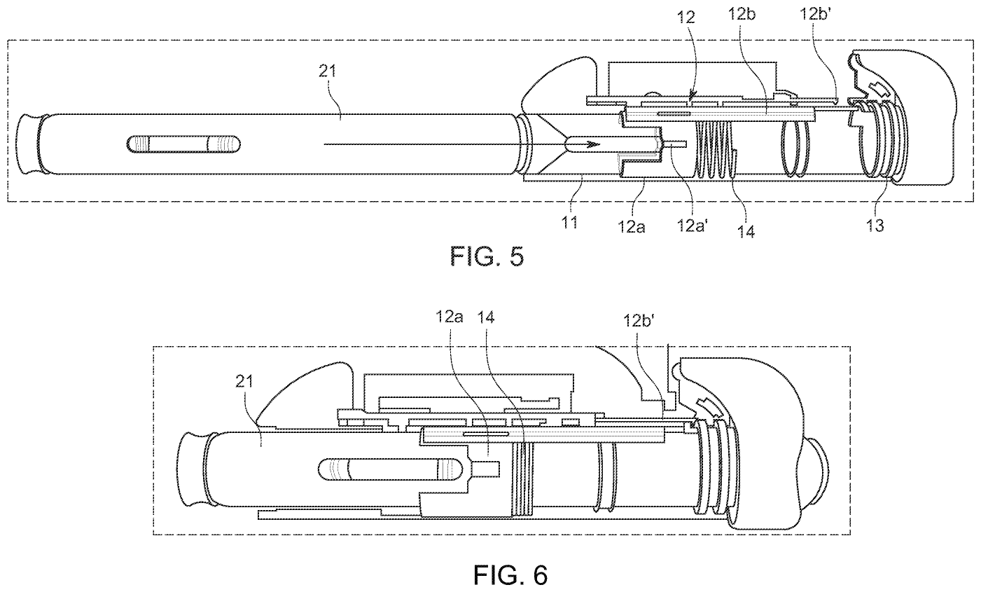

FIGS. 5-6 display a perspective view of the system when the medicament delivery device (2) is being positioned in the cavity of the monitoring unit (1) and when the medicament delivery device (2) is positioned and secured in the cavity of the monitoring unit (1).

The enclosing member (12a) comprises an engagement structure is configured to releasably engage with the engagement member (21a) on the medicament delivery device (2) when the medicament delivery device (2) is in the secure state.

In a preferred embodiment, the engagement structure (12a′) is a flexible structure, and configured to flex radially outward in its relaxed configuration; and to flex radially inward in its tensioned configuration. The inner surface of the body comprises a blocking structure e.g. a wall, configured to receive the engagement structure (12a′), tension, and keep it into its tensioned configuration. In another embodiment, the engagement structure (12a′) can be an axial stop rib configured to interact with the engagement member (21a) on the medicament delivery device (2) for axially locking the medicament delivery device (2) with the biased holding unit (12), wherein the axial stop rib is in the form of a wedge shape structure. In a further embodiment, the engagement structure (12a′) is a radial stop rib configured to interact with the engaging member (21a) on the medicament delivery device (2) for radially locking the medicament delivery device (2) with the biased holding unit (12).

The locking element (12b) and the enclosing member (12a) are fixed together, which can be a snap-fit or screw-fit, or they can also be integral i.e. one piece. The locking element (12b) further comprises a locking arm (12b′) extending distally whereby the distal end of the locking arm (12b′) is formed as a wedge shape structure or as a hook; and a rotational stop (not showed) arranged on the outer surface of the locking element (12b). The rotational stop is configured to engage a counter rotational stop on the inner surface of the body, so that the locking element (12b) is rotationally fixed in relation to the body. The rotational stop and the counter rotational stop can be a pair of grooves or guide tracks arranged on either the outer surface of the locking element (12b) or the inner surface of the body, and a spline arranged on the other outer surface of the locking element (12b) or the inner surface of the body; or a protrusion and a counter protrusion outer surface of the locking element (12b) or the inner surface of the body.

The counter locking element (13) is arranged to the movable portion (11a). The counter locking element (13) is received in the movable portion (11a). The counter locking element (13) and the movable portion (11a) are rotationally fixed together i.e. in relation to each other, or these parts can also be integral i.e. one piece, so that the counter locking element (13) and the movable portion (11a) can be rotated together in relation to the body (11). In one embodiment, a space is defined between the outer surface of the counter locking element (13) and the inner surface of the movable portion (11a). The outer surface of the counter locking element (13) further comprises a ledge arranged to receive the locking arm (12b′).

The biased holding unit (12) is axially slidable and rotationally locked in relation to the body (11) between the second position in which the enclosing member (12a) together with the locking element (12b) are pressed distally against the force of the biasing element (14) and is releasably connected to the body; and the first position in which the enclosing member (12a) together with the locking element (12b) are biased proximally forward to its most-forward position under the force of the biasing element (14).

When the biased holding unit (12) is in the second position, the locking arm (12b′) of the locking element (12b) is received in the space between the outer surface of the counter locking element (13) and the inner surface of the movable portion (11a), and is engaged with the ledge of the counter locking element (13), so that the biased holding unit is releasably connected to the body (11) since the counter locking element (13) and the movable portion (11a) are connected to the body

Once the locking arm (12b′) is disengaged from the counter locking element (13) by a rotation or pull force of the movable portion (11a) exerted by a user, the biased holding unit will move to the first position under the force of the biasing element (14) until the locking element and the enclosing member is stopped by an abutment arranged on the inner surface of the body (11) which is the most-forward position of the biased holding unit (12).

The monitoring device (1) is configured to receive and to releasably attach the medicament delivery device (2) to its body, in the secure state. The medicament delivery device (2) is received and releasably attached to the body (11) of the monitoring device (1) through the biased holding unit (12). To releasably attach the medicament delivery device (2) to the monitoring device (1) as illustrated in FIG. 5-6, the medicament delivery device (2) needs to be axially positioned in the cavity and passed through the enclosing member (12a) when the biased holding unit (12) is in the first position and the engagement structure (12a′) is in its relax configuration and proximally arranged to the blocking wall. Once the medicament delivery device (2) is pressed into the cavity towards the distal direction, such like the user might get the monitoring device (1) on one hand and press the injection device (1) into the cavity by the other hand; or the user might press the injection device (1) into the cavity by pressing on a flat surface, e.g. surface of table or floor; the engagement member (21a) of the medicament delivery device (2) will abut a surface of the enclosing member (12a) such that the biased holding unit (12) is moved towards the distal direction; and once the engagement structure (12a′) pass through the blocking wall, the blocking wall will bend the engagement structure (12a′) flexing radially inward into its tensioned configuration, so that the engagement structure (12a′) now starts to cramp on the engagement member (21a) of the medicament delivery device (2). Both the enclosing member and the locking element (12b) are moved towards the distal direction and once the locking arm (12b′) passed the ledge on the outer surface of the counter locking element (13), the engagement structure (12a′) start to tightly cramp the engagement member (21a) of the medicament delivery device (2). When the locking arm (12b′) passes the ledge on the outer surface of the counter locking element (13), it generates a click sound as an indication that the medicament delivery device is in the secure state. Since the engagement structure (12a′) is tightly cramping the medicament delivery device (2), the medicament delivery device (2) is releasably attached to the monitoring device (1).

For releasing the medicament delivery device from the monitoring device (2), the movable portion (11a) can be twisted or pulled. Moving the movable portion (11a) causes the counter locking element (13) to be moved at the same time such that the ledge on the outer surface of the counter locking element (13) will then misalign with the locking arm (12b′) and the biased holding unit is moved from its second position into the first position under the biasing force from the biasing element (14). Once the engagement structure (12a′) fully passes the blocking wall in the proximal direction, the engagement structure (12a′) is moved into its relaxed configuration under its own radially resilient force; thereby the engagement member (21a) of the medicament delivery device (2) is no longer cramped by the flexible arm (12a′), and thus the medicament delivery device (2) is in the unsecure state and can be released from the monitoring device (1).

The counter locking element (13), in an alternative embodiment, can be the ledge itself and can be arranged on the inner surface of the movable portion (11a), and configured to engage or disengage with the locking arm (12b′).

The concept of this disclosure has mainly been described above with reference to a few examples. However, as is readily appreciated by a person skilled in the art, other embodiments than the ones disclosed above are equally possible within the scope of this disclosure, as defined by the appended claims.

1-15. (canceled)

16. A system comprising a monitoring device and a medicament delivery device wherein the monitoring device is configured to be releasably attached to the medicament delivery device and to monitor an operation of the medicament delivery device, wherein the monitoring device comprises:
a body defining a cavity for receiving the medicament delivery device;
a biased holding unit arranged in the body and configured to retain the medicament delivery device to the body in a secure state;
wherein the biased holding unit is movable within the body between a first position in which the medicament delivery device is in an unsecure state and a second position in which the medicament delivery device is in the secure state;
wherein the biased holding unit comprises an enclosing member having an engagement structure;
and in that the biased holding unit further comprises a locking element fixed to or integral with the enclosing member,
wherein the engagement structure is configured to interact with an engagement member on the medicament delivery device and the locking element is configured to interact with a counter locking element arranged to the body when the biased holding unit is in the second position.

17. The system according to claim 16, wherein a biasing element is positioned between the biased holding unit and the body.

18. The system according to claim 17, wherein the biasing element is configured to be in a compressed state when the biased holding unit is in the second position, and in a released state when the biased holding unit is in the first position.

19. The system according to claim 16, wherein the engagement structure is a flexible structure element.

20. The system according to claim 16, wherein the engagement structure is configured to be releasably engaged with the engagement member on the medicament delivery device when the medicament delivery device is in the secure state.

21. The system according to claim 16, wherein the body comprises a movable portion.

22. The system according to claim 21 wherein the movable portion is twistable in relation to a longitudinal axis.

23. The system according to claim 21, wherein the counter locking element is arranged to the movable portion of the body.

24. The system according to claim 16, wherein the biased holding unit is rotationally locked and slidable in relation to the body between a first position and a second position.

25. The system according to claim 16, wherein the locking element further comprises a locking arm extending distally and whereby the distal end of the locking arm is formed as a wedge shape structure or as a hook.

26. The system according to claim 16, wherein the counter locking element comprises a ledge arranged to receive the locking arm.

27. The system according to claim 16, wherein the engagement structure is an axial stop rib configured to interact with the engagement member on the medicament delivery device for axially locking the medicament delivery device with the biased holding unit.

28. The system according to claim 16, wherein the engagement structure is a radial stop rib configured to interact with the engaging member on the medicament delivery device for radially locking the medicament delivery device with the biased holding unit.

29. The system according to claim 26, wherein the axial stop rib is a wedge shape structure.

30. The system according to claim 16, wherein the medicament delivery device is an injection device.

31. A system comprising a monitoring device and a medicament delivery device having a longitudinal axis, where the monitoring device is configured to be releasably attached to the medicament delivery device and monitors an operation of the medicament delivery device,
wherein the monitoring device comprises:
a body defining a cavity for receiving the medicament delivery device and having a movable portion;
a biased holding unit arranged in the body and configured to retain the medicament delivery device to the body in a secure state;
wherein the biased holding unit is movable within the body between a first position in which the medicament delivery device is in an unsecure state and a second position in which the medicament delivery device is in the secure state,
wherein the biased holding unit comprises an enclosing member having an engagement structure and a locking element fixed to, or integral with, the enclosing member, and
wherein the engagement structure is configured to interact with an engagement member on the medicament delivery device and the locking element is configured to interact with a counter locking element operatively connected to the movable portion of the body when the biased holding unit is in the second position.

32. The system according to claim 31, wherein twisting of the movable portion relative to the longitudinal axis causes rotational movement of the counter locking element.

33. The system according to claim 31, wherein the locking element further comprises a locking arm extending distally and operatively engages the counter locking element.

34. The system according to claim 33, wherein a distal end of the locking arm is formed as a wedge shape structure or as a hook.

35. The system according to claim 33, wherein the counter locking element comprises a ledge arranged to receive a distal end of the locking arm.
